(19) United States
(12) Patent Application Publication
(10) Pub. No.: US 2016/0045699 A1
(43) Pub. Date: Feb. 18, 2016

(54) Fiberoptic Bronchoscopy Intubation Device and Method of Using the Same

(71) Applicant: Richard Cardillo, Fort Wayne, IN (US)

(72) Inventor: Richard Cardillo, Fort Wayne, IN (US)

(21) Appl. No.: 14/459,634

(22) Filed: Aug. 14, 2014

(51) Int. Cl. A61M 16/04; A61B 1/07; A61B 1/267

(52) U.S. Cl. CPC A61M 16/0493; A61B 1/2676; A61B 1/07; A61M 16/0497

(57) ABSTRACT

A fiberoptic orotracheal intubation apparatus, said apparatus comprising: an oropharyngeal airway, said airway comprising a proximal end, a distal end, a top surface, a lingual surface, and a plurality of receiving tabs forming a central channel for receiving a desired elongated device and a parallel channel on each side of said central channel, each of said outer channels sized to receive and hold an elongated tubular medical instrument; a cannulated bite block, said bite block selectively attached to said proximal end of said airway and comprising a pair of opposing flanges disposed at the proximal end of said bite block and extending laterally from said bite block; and a flexible retaining strap having a first and second end, said first end of said retaining strap releasably attached to one of said opposing flanges, and said second end of said retaining strap releasably attached to one of said opposing flanges.

### BACKGROUND

1. Field of the Art

The present invention is generally related to medical devices. More specifically, the present invention is related to an intubation device for insertion to the endotracheal tube of a human patient during a bronchoscopy or other medical procedure where a patient needs a temporary artificial airway.

2. Description of the Prior Art

Fiberoptic bronchoscopy (“FOB”) is one of the most important methods of intubating patients with difficult airways. For circumstances in which it is desirable to perform this via the oral route, specific airways have been devised to push the tongue anteriorly to clear a passage for the fibre scope into the trachea. Although some of these airways were originally proposed for use in awake patients, they are now predominantly used in anaesthetized patients.

Of these airways, the most widely known are the Williams airway intubator (Williams Airway Intubator Ltd, Calgary, Canada), the Ovassapian Fibreoptic Intubating Airway (Kendall, Argyle, New York, N.Y., USA) and the Berman Oropharyngeal Airway (Hsin Kuang Yeh Co. Ltd., Taichung Hsien, Taiwan). These devices unfortunately do nor provide a reliable means of controlled ventilation and therefore should be distinguished from ‘dedicated airways’ including the Cobra PLA (Engineered Medical System, Indianapolis. Ind., USA), the Cuffed Oropharyngeal Airway (Mallinckrodr Medical Inc, St Louis, Mo., USA), and the LMA-ProSeal (The Laryngeal Mask Company Led, Maidenhead, UK).

So called “non dedicated” airways are dedicated to the maintenance of airway patency while other major airway interventions arc anticipated or are in progress; thus, they must be free of any significant procedural delay and, therefore, they must be highly reliable as air conduits. Additionally, a non dedicated airway is needed that doesn't suffer from some of the known deficiencies in the prior art. For example, the Williams airway intubator cannot easily be removed after intubation unless the tracheal tube connector is removed, and if the airway is left in place too long, upper airway trauma and ulceration may result. Additionally, the inner surface needs to be well lubricated to allow the tracheal tube to slide along the curvature of the airway. Damage of the tracheal cuff is a frequent problem if a large tracheal tube is used with inadequate lubrication. Moreover, the inflexible plastic of most prior art airways often does not conform to the patient's airway after insertion, and this fact may lead to malpositioning during bronchoscopy, which can lead to impaction into the tongue base or dental damage. In some alternative medical techniques, the airway is removed after the FOB is positioned in the trachea. Unfortunately, this later method means that the function of the airway is lost during the tracheal tube insertion and the tube may impinge on supraglottic structures. Examples of these limitations are shown in the prior art references below:

U.S. Pat. No. 377,622 discloses an entubator for an endotracheal tube which includes a coherent flexible fiber optic bundle which can be positioned within the central cavity or lumen of the endotracheal tube for viewing the anatomy of a patient as the endotracheal tube advances through the patient during the insertion procedure. The entubator also includes a means for directing the course of travel of the endotracheal tube which enables the operator to navigate the endotracheal tube through anatomical passageways made visible by the fiber optics bundle. The fiber optic bundle and the means for directing the course of travel enable the entubation of an endotracheal tube through the nasopharynx of the patient. Method of entubing an endotracheal tube through the nasopharynx.

U.S. Pat. No. 4,041,936 discloses a tube especially useful in fiberoptic bronchoscopy has at one end an adapter which is provided with both means for connection to ventilating means and means for introduction of a fiberoptic bronchoscope and has at the other end a section of reduced external diameter provided with an inflatable cuff.

U.S. Pat. No. 4,580,556 discloses an adaptor is disclosed for use with an anesthesia mask to allow an endotracheal tube and a fiberoptic laryngoscope to be passed through the mask while a patient is being ventilated under general anesthesia. The adaptor comprises a T-shaped member, one limb of which is connected to conventional anesthesia equipment and the other limb of which has a soft, flexible cover with a central opening through which the endotracheal tube and fiberoptic laryngoscope are passed. The flexibility of the cover allows endotracheal tubes of different diameter to be accommodated in the opening, and when the mask is not being used for intubation, the opening in the cover may be obturated by a plug. The adaptor may also include a further plug having a throughbore for insertion in the free end of the endotracheal tube to seal around the laryngoscope.

U.S. Pat. No. 4,683,874 discloses a bronchofiberscope is provided that contains a sterile and uncontaminated distal end of an insertion tube with sleeves and a disolvable membrane over it to facilitate the distal end past vocal cords so that the sleeves can be pulled back releasing the disolvable membrane to expose the distal end whereby viewing of a windpipe and the branches is done with a light guide and an objective lens and removal of bronchial secretions is facilitated through a suction/forceps channel to remove an uncontaminated specimen for an appropriate bacteriologic examination.

U.S. Pat. No. 5,016,614 discloses a medical instrument for facilitating endotracheal intubation in a patient, the instrument including a handle and an elongated obturator element extending therefrom, the obturator element for releasably retaining thereon a selected endotracheal tube for positioning within the patient, a mechanism being provided for selectively retaining and ejecting the endotracheal tube from the obturator element, the obturator element accommodating there through an endoscope to permit visualization at the end thereof to facilitate placement of the endotracheal tube, the apparatus also including structure for providing controlled suction at the end of the obturator element.

U.S. Pat. No. 5,429,118 discloses a disposable sheath for a medical scope and having a flexible, elongated body portion formed from a translucent plastic material with a rough-textured interior surface and a flexible, transparent tip portion. The rough-textured interior surface includes projections that result from a plurality of indentations formed in the exterior surface of the body portion of the sheath. The translucent plastic material of the body portion is bonded to the transparent tip portion of the sheath by high frequency heat sealing, which is pathogen impermeable. The plastic materials of the sheath are also pathogen impermeable. In one embodiment, the tip portion of the sheath is formed from a flat, foldable sheet of plastic material. The lateral edges of the foldable sheet are heat sealed to each other, and the proximal edges are heat sealed to the distal end of the body portion of the sheath. The body portion is formed from two strips of plastic material with a shape memory for a flat configuration that are longitudinally heat sealed along the edges thereof. A stylet is positioned in the sheath for maintaining the sheath in an open condition during handling and storage.

U.S. Pat. No. 5,431,152 discloses an endotracheal intubating instrument has an elongate curvilinear blade member releasably attachable to a handle and a central channel sized to removably receive and slidably engage an endotracheal tube therein, an elongate tubular housing removably connected at its forward end to the rearward end of the endotracheal tube which removably receives a fiberoptic scope having an eyepiece at a rearward end and a fiberoptic bundle extending forwardly within the endotracheal tube, and an adjustable positioning element through which the forwardly extending fiberoptic bundle passes for adjustably positioning and maintaining the tip end of the fiberoptic bundle relative to the forward end of the endotracheal tube. The apparatus is placed in the mouth, the larynx is identified, and the endotracheal tube, housing, and fiberoptic scope are advanced as a unit into the trachea as the blade is removed. Then the fiberoptic scope and housing are withdrawn, leaving the endotracheal tube in the desired part of the trachea.

U.S. Pat. No. 5,607,386 discloses a malleable fiberoptic intubating apparatus has an elongate arcuate thin-walled tubular stylet which removably carries an endotracheal tube around it and the fiberoptic bundle of a flexible fiberoptic bronchoscope within it, a handle at the rearward end of the stylet, and a telescoping bronchoscope support arm that releasably receives and carries a bronchoscope and moves relative to the handle. An adjustable endotracheal tube positioning element engages the proximal end of the endotracheal tube and positions it on the stylet. An adjustable fiberoptic bundle positioning element engages the flexible fiberoptic bundle of the bronchoscope and positions it within the stylet. The instrument is placed in the mouth, the larynx is identified, and the instrument is advanced as a unit into the trachea. Then the fiberoptic bronchoscope and stylet are removed from the mouth, leaving the endotracheal tube in the proper part of the trachea.

U.S. Pat. No. 5,800,342 discloses an endoscopic instrument for controlled introduction of tubular members in the body includes a blade assembly having a blade for being introduced in the body to retract or manipulate anatomical tissue for introduction of a tubular member at a site in the body, a tubular member assembly having a tubular member with a distal end for being introduced at the site in the body and a remote viewing assembly having a distal end for being positioned in the body to permit introduction of the tubular member at the site in the body to be confirmed visually at a location remote from the site in the body. The distal end of the tubular member and the distal end of the remote viewing device are movable along the blade to introduce the tubular member distal end at the site in the body. A method of introducing a tubular member at a site in the body includes the steps of inserting a blade of an instrument in the body, retracting anatomical tissue with the blade, moving a distal end of a tubular member of the instrument distally along the blade to introduce the distal end of the tubular member at the site in the body and visualizing introduction of the tubular member at the site in the body with a remote viewing device from a location remote from the site in the body.

U.S. Pat. No. 5,913,816 discloses an intubation device comprises an imaging conduit including an illumination conduit for delivering illumination to a distal end of the device and an image-receiving conduit for communicating images from the distal end of the device to the proximal end of the device. The device also comprises a flushing conduit for delivery of fluids and/or gases to the distal end of the device. The image conduit and the flushing conduit are attached at the distal end of the device. The distal end of the device can be manipulated by advancing the imaging conduit in an axial direction while maintaining the flushing conduit fixed in place. Furthermore, a length adjustment member is provided for adjusting the extent to which the distal end of the device extends beyond a distal end of an endotracheal tube which is inserted over the imaging and flushing conduits. The illuminating conduit and the image-receiving conduit terminate in separate ports at the distal end of the device. Furthermore, a substantial portion of the device is disposable, so that time consuming sterilization is not necessary.

U.S. Pat. No. 6,086,529 discloses an apparatus and method for accomplishing simultaneous bronchoscopy and airway instrumentation and securing the instrument in place using an adjustable threaded gastight seal. The apparatus consisting of a hollow elongated manifold for simultaneous fiberoptic bronchoscopy through a fiberoptic port and introduction a secondary tubular instrument through a threaded secondary instrument port while maintaining mechanical ventilation of a intubated patient using a ventilation port and endotracheal tube connection port. The secondary instrument port is constructed in a manner to allow introduction of a tubular instrument into the airway and form an adjustable gastight seal. It consists of a compressible perforated diaphragmatic seal sandwiched between a threaded secondary instrument port and threaded perforated secondary instrument cap so that the threaded cap may be torqued into the threaded secondary port compressing the compressible diaphragmatic seal against the inserted secondary instrument forming airtight adjustable seal and affixing the secondary instrument in place. In addition, the secondary instrument port allows chronic instrumentation following the removal of the fiberoptic bronchoscope or reinsertion.

U.S. Pat. No. 6,251,069 discloses a laryngoscope having a flexible blade which is hinged and having in the handle articulation control for the hinged portion of the blade as well as an actuator for a pair of balloons on the blade connected with double-lumen tubes.

U.S. Pat. No. 6,585,639 discloses an apparatus, methods, and kits, provided for use in combination with a conventional bronchoscope or other lung viewing scope. In particular, sheaths having an inflatable cuff at their distal end are provided to receive a viewing scope through a lumen thereof. The sheaths thus provide an inflatable cuff on a viewing scope assembly so that the scope can be used in procedures which require selective isolation of regions of a patient's lungs. In particular embodiments, the sheaths may include stop elements for properly positioning a viewing scope therein, pressure transducers for providing accurate lung pressure information during procedures, and the like. Methods for using and forming sheaths having inflatable cuffs are also described.

U.S. Pat. No. 747,329 discloses a flexible fiber optic bronchoscope with one-way valve is provided in which a unidirectional flow control valve is inserted between the bronchoscopic manifold port and the sealing cap. A compressible diaphragmatic seal with radial slits separate resiliently returnable valve flaps that deformably compress around the shaft of any inserted conduit. A circumscribing housing for typically affixing along a lower receiving ring to the bronchoscopic manifold port to which the sealing cap would normally be attached. An upwardly extending, annular attachment protuberance thereby provides the attachment for the conventional sealing cap.

U.S. Pat. No. 7,909,758 discloses a laryngoscope blade, wherein the blade comprises a tube having an opening along one side. This invention further provides a laryngoscope blade comprising at its front portion a first surface for lifting the epiglottis of a patient and a second surface for fixing the portion of the posterior part of the vocal cords of the patient.

U.S. Pat. No. 7,963,286 discloses a combination bite block, tongue depressor/retractor and airway for establishing and maintaining an open airway while preventing emergence clenching and the resulting dental and soft tissue damage associated with emergence clenching in procedures where anesthesia and/or sedation are used, or when the patient is not in control of their own airway, regardless of the cause. The inventive subject matter includes a tongue depressor/retractor component in both right and left conformations; and a bite block component. The bite block component is a wedge shaped, compressible component that is inserted between the upper and lower molars on either the right or left side of the mouth. The tongue depressor component is comprised by a flat portion of that is inserted into the side of the bite block, and an optional curved portion that retracts the tongue off of the posterior pharynx when in place.

U.S. Pat. No. 8,704,467 discloses a rapid orotracheal intubation guide device is disclosed that facilitates orotracheal intubation or direct orotracheal visualization without resting the device on the subject's tongue and that, during a bronchoscopic intubation, automatically ejects the endotracheal tube from the device as the endotracheal tube is advanced coaxially over a bronchoscope previous placed through the guide, thereby providing a mid-line entry of the bronchoscope and endotracheal tube, higher intubation success rate by lower skilled operators, and easy removal of the device from the patient's oral cavity.

U.S. Pat. No. 8,766,967 discloses an intubation a movable guide having a guide extension, and a guide tip; at least one trachea condition sensor to detect a trachea condition wherein the at least one trachea condition sensor is disposed on the guide; and a guide control device operatively coupled to the guide, the guide control device to move the guide in response to the trachea condition.

Thus, as shown in the above referenced patents, a need exists for a fiberoptic bronchoscopy intubation device that overcomes the limitations of prior art devices.

### SUMMARY

In one exemplary embodiment, the present invention comprises a fiberoptic orotracheal intubation apparatus, having an oropharyngeal airway, said airway comprising a proximal end, a distal end, a top surface, a lingual surface, and a plurality of receiving tabs forming a central channel for receiving a desired elongated device and a parallel channel on each side of said central channel, each of said outer channels sized to receive and hold an elongated tubular medical instrument; a cannulated bite block, said bite block selectively attached to said proximal end of said airway and comprising a pair of opposing flanges disposed at the proximal end of said bite block and extending laterally from said bite block; and a flexible retaining strap having a first and second end, said first end of said retaining strap releasably attached to one of said opposing flanges, and said second end of said retaining strap releasably attached to one of said opposing flanges.

In another exemplary embodiment, the present invention comprises a fiberoptic orotracheal intubation apparatus system, said system comprising: at least one oropharyngeal airway, each of said airways comprising a specific size, a proximal end, a distal end, a top surface, a lingual surface, and a plurality of receiving tabs forming a central channel for receiving a desired elongated device and a parallel channel on each side of said central channel, each of said outer channels sized to receive and hold an elongated tubular medical instrument at least one cannulated bite block, each of said bite blocks comprising a specific size and selectively attached to said proximal end of said airway, each of said bite blocks further comprising a pair of opposing flanges disposed at the proximal end of said bite block and extending laterally, from said bite block; a flexible retaining strap having a first and second end, said first end of said retaining strap releasably attached to one of said opposing flanges, and said second end of said retaining strap releasably attached to one of said opposing flanges; and at least one elongated medical instrument.

In still another exemplary embodiment, the present invention comprises a method of inserting a fiberoptic orotracheal intubation apparatus, said method comprising the steps of: selecting an oropharyngeal airway, said airway comprising a specific size, a proximal end, a distal end, a top surface, a lingual surface, and a plurality of receiving tabs forming a central channel for receiving a desired elongated device and a parallel channel on each side of said central channel, each of said outer channels sized to receive and hold an elongated tubular medical instrument; selecting a cannulated bite block, said bite block comprising a specific size and selectively attached to said proximal end of said airway, each of said bite blocks further comprising a pair of opposing flanges disposed at the proximal end of said bite block and extending laterally from said bite block; assembling the bite block to the proximal end of the airway; attaching a flexible strap to the flanges of the bite block; inserting the airway into a patient's throat; and placing the flexible retaining strap around the head of the patient.

### BRIEF DESCRIPTION OF THE DRAWINGS

The present invention will be understood more fully from the detailed description given hereinafter and from the accompanying drawings of the preferred embodiment of the present invention, which, however, should not be taken to limit the invention, but are for explanation and understanding only.

In the drawings:

FIG. 1 shows an intubation device according to the present invention.

FIG. 2 shows a perspective assembly view of an intubation device according to the present invention.

FIG. 3 shows a perspective assembly view of an intubation device according to the present invention.

### DETAILED DESCRIPTION OF THE EMBODIMENTS

The present invention will be discussed hereinafter in detail in terms of the preferred embodiment according to the present invention with reference to the accompanying drawings. In the following description, numerous specific details are set forth in order to provide a thorough understanding of the present invention. It will be obvious, however, to those skilled in the art that the present invention may be practiced without these specific details. In other instance, well-known structures are not shown in detail in order to avoid unnecessary obscuring of the present invention.

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations.

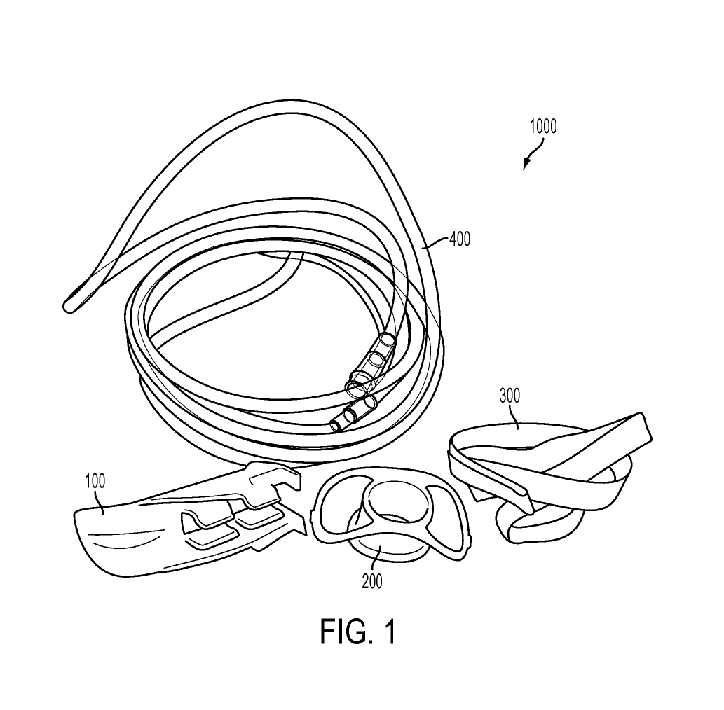

All of the implementations described below are exemplary implementations provided to enable persons skilled in the art to make or use the embodiments of the disclosure and are not intended to limit the scope of the disclosure, which is defined by the claims. In the present description, the terms “upper”, “lower”, “left”, “rear”, “right”, “front”, “vertical”, “horizontal”, and derivatives thereof shall relate to the invention as oriented in FIG. 1.

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

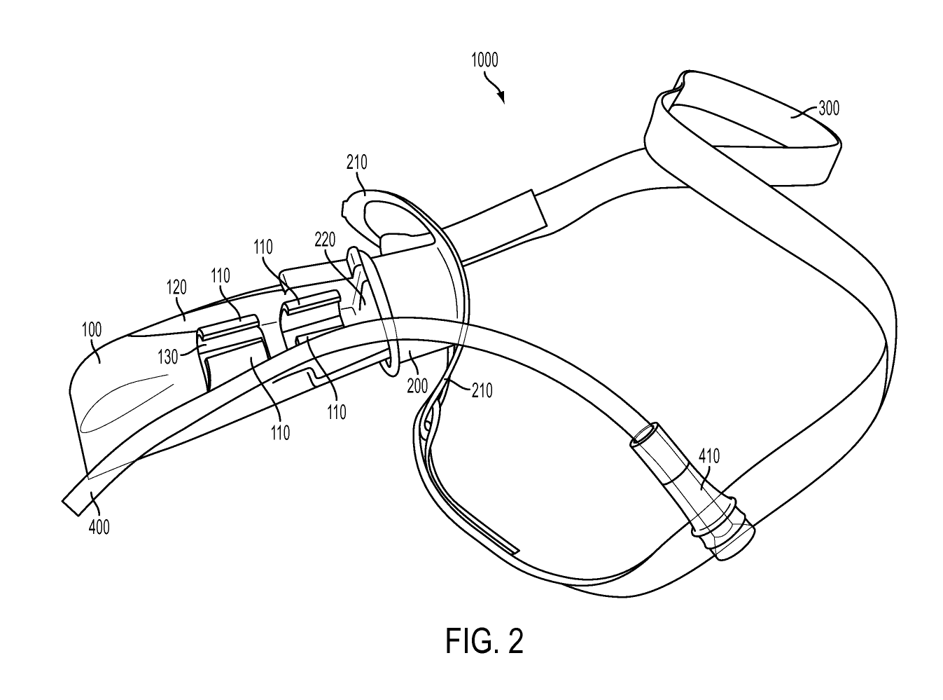

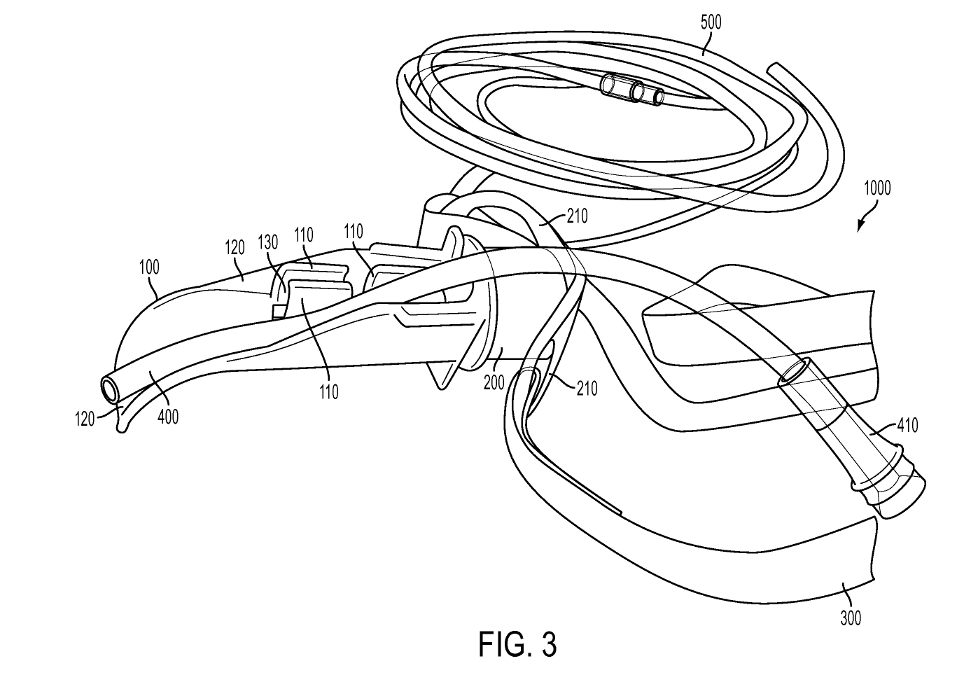

Referring first to FIG. 1, there is shown a disassembled view of fiberoptic intubation device 1000. As illustrated in FIG. 1, device 1000 generally comprises oropharyngeal airway 100, bite block 200, retaining strap 300, and elongated tubular medical instrument 400. These several components are described in greater detail below with reference to FIGS. 2 and 3.

Referring now to FIG. 2, there is shown a perspective assembly view of intubation device 1000. As illustrated in FIG. 2, intubation device 1000 comprises airway 100. Airway 100 is preferably comprised of a plastic material suitable for use in the human body. Airway 100 further comprises a two or more sets of tabs 110. Tabs 110 are disposed along a central longitudinal axis of airway 100 to form channel 130. Path 130 is collinear with the bite block 200. Airway 100 further comprises a pair of channels 120 on either side of central channel 130.

As further illustrated in FIG. 2 and in FIG. 3, intubation device 1000 further comprises bite block 200. Bite block 200 comprises a plastic material suitable for use in the human body. Bite block 200 further comprises a central bore 220, and a pair of laterally extensions 210. Each of said lateral extensions 210 is connected to one end of flexible strap 300. Flexible strap 300 is used to hold intubation device 1000 around a patient's head while the device is in use.

Referring still to FIG. 2 and to FIG. 3, any of channels 130 or 120 may be used to hold elongated medical devices such as air tube 400, or tubular illumination devices.

The above-described embodiments are merely exemplary illustrations set forth for a clear understanding of the principles of the invention. Many variations, combinations, modifications, or equivalents may be substituted for elements thereof without departing from the scope of the invention. It should be understood, therefore, that the above description is of an exemplary embodiment of the invention and included for illustrative purposes only. The description of the exemplary embodiment is not meant to be limiting of the invention. A person of ordinary skill in the field of the invention or the relevant technical art will understand that variations of the invention are included within the scope of the claims.

1. A fiberoptic orotracheal intubation apparatus, said apparatus comprising:
an oropharyngeal airway, said airway comprising a proximal end, a distal end, a top surface, a lingual surface, and a plurality of receiving tabs forming a central channel for receiving a desired elongated device and a parallel channel on each side of said central channel, each of said outer channels sized to receive and hold an elongated tubular medical instrument;
a cannulated bite block, said bite block selectively attached to said proximal end of said airway and comprising a pair of opposing flanges disposed at the proximal end of said bite block and extending laterally from said bite block; and
a flexible retaining strap having a first and second end, said first end of said retaining strap releasably attached to one of said opposing flanges, and said second end of said retaining strap releasably attached to one of said opposing flanges.

2. A fiberoptic orotracheal intubation apparatus system, said system comprising: at least one oropharyngeal airway, each of said airways comprising a specific size, a proximal end, a distal end, a top surface, a lingual surface, and a plurality of receiving tabs forming a central channel for receiving a desired elongated device and a parallel channel on each side of said central channel, each of said outer channels sized to receive and hold an elongated tubular medical instrument; at least one cannulated bite block, each of said bite blocks comprising a specific size and selectively attached to said proximal end of said airway, each of said bite blocks further comprising a pair of opposing flanges disposed at the proximal end of said bite block and extending laterally from said bite block; a flexible retaining strap having a first and second end, said first end of said retaining strap releasably attached to one of said opposing flanges, and said second end of said retaining strap releasably attached to one of said opposing flanges; and at least one elongated medical instrument.

3. A method of inserting a fiberoptic orotracheal intubation apparatus, said method comprising the steps of: selecting an oropharyngeal airway, said airway comprising a specific size, a proximal end, a distal end, a top surface, a lingual surface, and a plurality of receiving tabs forming a central channel for receiving a desired elongated device and a parallel channel on each side of said central channel, each of said outer channels sized to receive and hold an elongated tubular medical instrument; selecting a cannulated bite block, said bite block comprising a specific size and selectively attached to said proximal end of said airway, each of said bite blocks further comprising a pair of opposing flanges disposed at the proximal end of said bite block and extending laterally from said bite block; assembling the bite block to the proximal end of the airway; attaching a flexible strap to the flanges of the bite block; inserting the airway into a patient's throat; and placing the flexible retaining strap around the head of the patient.
